(19) United States
(12) Patent Application Publication
(10) Pub. No.: US 2011/0275857 A1
(43) Pub. Date: Nov. 10, 2011

(54) Antibody-Guided Fragment Growth

(75) Inventor: Alastair David Griffiths Lawson, Berkshire (GB)

(73) Assignee: UCB PHARMA S.A., Brussels (BE)

(21) Appl. No.: 13/143,747

(22) PCT Filed: Jan. 12, 2010
(86) PCT No.: PCT/GB2010/000040

§ 371 Date: Jul. 8, 2011

(30) Foreign Application Priority Data
Jan. 12, 2009 (GB) 0900425.0

(51) Int. Cl. C07C 233/00; C07C 39/00; G01N 33/68

(52) U.S. Cl. 564/123; 436/501; 568/716

(57) ABSTRACT

The present invention relates to an improved method for drug discovery comprising using contact residue information derived from antibody-protein target interactions to help to direct the growth of small molecule fragments during the synthesis of a drug candidate. In particular, the present invention relates to the use of atomic structural information derived from anti-body-protein interactions to guide the growth of small molecular fragments during lead optimisation, thus generating small molecule compounds which can alter the biological activity of a target protein.

The present invention relates to an improved method for drug discovery comprising using contact residue information derived from antibody-protein target interactions to help to direct the growth of small molecule fragments during the synthesis of a drug candidate.

In particular, the present invention relates to the use of atomic structural information derived from antibody-protein interactions to guide the growth of small molecular fragments during lead optimisation, thus generating small molecule compounds which can alter the biological activity of a target protein. The present invention also relates to the therapeutic uses of the compounds identified.

Screening of small molecule or compound fragments has rapidly gained acceptance in the pharmaceutical industry as a means of generating chemical hits. Fragment-based drug discovery is focused on binding efficiency rather than potency alone and the fragments themselves can be used as the initial building blocks for the development of a new drug. Also due to their smaller size, smaller libraries of compounds need to be screened compared to libraries of larger compounds in order to identify compounds that bind to the target protein. Typically once fragments which bind a target protein have been identified, structural information about where the fragments are binding on the protein is obtained from crystallographic studies. By using this information, fragments binding at adjacent sites may be further combined onto a template or individual fragments may be used as the starting point for growing out a higher molecular weight structure, for example, into other pockets on an active site (See Blundell et al., 2002, Nature Reviews, 1, 45-54).

The growth of compound fragments is currently restricted by the amount of structural information available for a target protein. Such structural information may include for example the active site or a receptor binding site on the target protein, in the presence or absence of a ligand, receptor and/or a small molecule inhibitor. Whilst this structural information may provide a useful guide to suitable regions on the target protein in which to target fragment growth, it does not necessarily provide pre-validated sites or contact atoms to which fragment growth may be directed. Compound fragment growth can therefore be an essentially random process of trial and error.

Hence there is a need in the art to provide improved methods for compound fragment growth.

Antibodies are very useful therapeutic agents given their antigen binding specificity and high affinity. For a given protein target it is possible to obtain function modifying antibodies which bind the target protein, each antibody potentially binding at a different site on the protein. To date structural information about antibody binding has been used to design peptide mimetics that mimic the antibody structure, see for example Park et al., 2000, Nature Biotechnology, 18, 194-198; Casset et al., 2003, Biochemical and Biophysical Research Communications 307, 198-205). In the present invention the structural information about where and how a function modifying antibody binds a target protein is used to provide pre-validated contact atoms on both the target protein and the antibody which can be used to guide compound fragment growth, thus enhancing the likelihood of a compound generated by antibody-guided fragment growth being an effective modulator of the activity of the target protein. Furthermore, the use of antibody contact information may direct compound synthesis to new, previously unexplored sites on the protein.

In one example, the present invention provides a method of generating a small molecule compound that can alter the activity of a target protein comprising:

a) obtaining one or more antibodies or fragments thereof which bind to the target protein and alter the biological activity of the target protein
b) generating a three-dimensional structural representation of an antibody obtained in step (a) in association with the target protein and identifying one or more pairs of contact atoms on the target protein and the antibody that interact with each other and which fall within a binding site of the antibody
c) obtaining one or more compound fragments that bind to the target protein
d) generating a three-dimensional structural representation of one or more of the fragments obtained in step (c) in association with the target protein
e) selecting a compound fragment which binds the target protein within or in the vicinity of the antibody binding site identified in step (b)
f) growing the compound fragment selected in step (e) to generate one or more candidate compounds which interact with one or more contact atoms on the target protein identified in step (b), optionally by directing the chemical growth of the compound fragment such that the expanded fragment occupies the same chemical space or 3D location as one or more of the contact atoms on the antibody identified in step (b)
g) testing one or more of the candidate compounds produced in step (f) for improved affinity for the target protein and/or improved potency and/or the ability to alter the biological activity of the target protein
h) selecting a candidate compound tested in step (g) if it modulates the activity of the target protein or has improved binding affinity or ligand efficiency
(i) optionally performing further chemistry and screening using the candidate compound identified in step (h) to generate a small molecule compound which modulates activity of the target protein.
   It will be appreciated that steps (a) to (d) need not necessarily be performed exactly in that order. In one example steps (a) and (c) may be performed before steps (b) and (d). In one example steps (a), (c) and (d) may be performed before step (b). In one example steps (c) and (d) may be performed before steps (a) and (b). It will also be appreciated that certain steps may be performed sequentially or in parallel where appropriate. For example, steps (a) and (c) may be performed in parallel and steps (b) and (d) may also be performed in parallel, after steps (a) and (c).

### Target Protein

A target protein of the present invention can be any kind of protein or polypeptide amenable to influence by the binding of another molecule. Typical categories of targets include, but are not limited to, enzymes, cytokines, receptors, transporters and channels. In certain embodiments the target protein is known to have a function in disease onset, development or establishment. In the present invention antibodies and compounds are identified which modulate the activity of the target protein in a desirable way, for example to inhibit the activity of the target protein or to stimulate the activity of the target protein.

The target polypeptide for use in the present invention may be the ‘mature’ polypeptide or a biologically active fragment or derivative thereof. Target polypeptides may be prepared by processes well known in the art from genetically engineered host cells comprising expression systems or they may be recovered from natural biological sources. In the present application, the term “polypeptides” includes peptides, polypeptides and proteins. These are used interchangeably unless otherwise specified. The target polypeptide may in some instances be part of a larger protein such as a fusion protein for example fused to an affinity tag. In some instances the target protein may be expressed naturally on the surface of a cell and cell surface expressed protein may be used, either as recombinant cells or naturally occurring cell populations.

It will be appreciated that the exact nature of the target protein used in the method of the present invention may vary at different stages of the method, for example fragments or domains or mutations of the target protein may be used in certain screens or structural representations where appropriate. In some cases these may not be biologically active.

Suitable screens for determining the activity of each target protein may be known in the art or can be devised experimentally. Such screens therefore allow the effect of an antibody or candidate compound on the activity of the target protein to be determined. Such screens include for example signalling assays, detecting receptor/ligand interactions or enzyme activity assays. It will be appreciated that each screen will depend on the nature of the target protein and that more than one screen may be used.

In the method of the present invention, candidate compounds, compound fragments or antibodies may each be tested for their affect on the biological activity of the target protein. For example, the antibodies and compounds identified which bind to the target protein may be introduced via standard screening formats into biological assays to determine the inhibitory or stimulatory activity of the compounds or antibodies, or alternatively or in addition, binding assays to determine binding or blocking, such as ELISA or BIAcore may be appropriate.

Examples of antibodies produced in the method of the present invention may include but are not limited to neutralising antibodies, antagonistic or agonistic antibodies. In one example therefore the antibodies identified in step (a) of the method alter the biological activity of the target protein by neutralising, antagonising or agonising the biological activity of the target protein. In one example therefore the antibodies identified in step (a) of the method alter the biological activity of the target protein by stimulating or inhibiting the biological activity of the target protein. In one example the antibody may be a ‘neutralising antibody’, an antibody that is capable of neutralising the biological activity of a given protein, such as signalling activity, for example by blocking binding of a target protein to one or more of its receptors. In such an example the small molecule compound generated by the method of the present invention may similarly block binding of the target protein to one or more of its receptors and neutralise the biological activity of the protein. As used herein with reference to the activity of the target protein the terms ‘modulate’ and ‘alter’ are used interchangeably.

### Antibodies

Function modifying antibodies for use in the present invention may be obtained using any suitable method known in the art. Preferably the function modifying antibodies obtained in step (a) of the method are generated using one or more of the methods described herein below.

The target polypeptide or cells expressing the target polypeptide can be used to produce antibodies which specifically recognise the target polypeptide. Antibodies generated against the target polypeptide may be obtained, where immunisation of an animal is necessary, by administering the polypeptides to an animal, preferably a non-human animal, using well-known and routine protocols, see for example Handbook of Experimental Immunology, D. M. Weir (ed.), Vol 4, Blackwell Scientific Publishers, Oxford, England, 1986). Many warm-blooded animals, such as rabbits, mice, rats, sheep, cows, pigs or camelids (e.g. camels, llamas) may be immunized.

Antibodies for use in the present invention include whole antibodies of any suitable class for example, IgA, IgD, IgE, IgG or IgM or subclass such as IgG 1, IgG2, IgG3 or IgG4, and functionally active fragments or derivatives thereof and may be, but are not limited to, monoclonal, humanised, fully human or chimeric antibodies.

Antibodies for use in the present invention may therefore comprise a complete antibody molecule having full length heavy and light chains or a fragment thereof and may be, but are not limited to Fab, modified Fab, Fab′, F(ab′)₂, Fv, single domain antibodies (such as VH, VL, VHH, IgNAR V domains), scFv, bi, tri or tetra-valent antibodies, Bis-scFv, diabodies, triabodies, tetrabodies and epitope-binding fragments of any of the above (see for example Holliger and Hudson, 2005, Nature Biotech. 23(9):1126-1136; Adair and Lawson, 2005, Drug Design Reviews—Online 2(3), 209-217). The methods for creating and manufacturing these antibody fragments are well known in the art (see for example Verma et al., 1998, Journal of Immunological Methods, 216, 165-181). Other antibody fragments for use in the present invention include the Fab and Fab′ fragments described in International patent applications WO2005/003169, WO2005/003170 and WO2005/003171. Multi-valent antibodies may comprise multiple specificities e.g. bispecific or may be monospecific (see for example WO 92/22853 and WO05/113605).

The term ‘antibody’ as used herein may also include binding agents which comprise one or more CDRs incorporated into a biocompatible framework structure. In one example, the biocompatible framework structure comprises a polypeptide or portion thereof that is sufficient to form a conformationally stable structural support, or framework, or scaffold, which is able to display one or more sequences of amino acids that bind to an antigen (e.g. CDRs, a variable region etc.) in a localised surface region. Such structures can be a naturally occurring polypeptide or polypeptide ‘fold’ (a structural motif), or can have one or more modifications, such as additions, deletions or substitutions of amino acids, relative to a naturally occurring polypeptide or fold. These scaffolds can be derived from a polypeptide of any species (or of more than one species), such as a human, other mammal, other vertebrate, invertebrate, plant, bacteria or virus.

Typically the biocompatible framework structures are based on protein scaffolds or skeletons other than immunoglobulin domains. For example, those based on fibronectin, ankyrin, lipocalin, neocarzinostain, cytochrome b, CP1 zinc finger, PST1, coiled coil, LACI-D1, Z domain and tendramisat domains may be used (See for example, Nygren and Uhlen, 1997, Current Opinion in Structural Biology, 7, 463-469).

The term ‘antibody’ as used herein may also include binding agents based on biological scaffolds including Adnectins, Affibodies, Darpins, Phylomers, Avimers, Aptamers, Anticalins, Tetranectins, Microbodies, Affilins and Kunitz domains.

Monoclonal antibodies may be prepared by any method known in the art such as the hybridoma technique (Kohler & Milstein, 1975, Nature, 256:495-497), the trioma technique, the human B-cell hybridoma technique (Kozbor et al., 1983, Immunology Today, 4:72) and the EBV-hybridoma technique (Cole et al., Monoclonal Antibodies and Cancer Therapy, pp 77-96, Alan R Liss, Inc., 1985).

Antibodies for use in the invention may also be generated using single lymphocyte antibody methods by cloning and expressing immunoglobulin variable region cDNAs generated from single lymphocytes selected for the production of specific antibodies by for example the methods described by Babcook, J. et al., 1996, Proc. Natl. Acad. Sci. USA 93(15):7843-78481; WO92/02551; WO2004/051268 and International Patent Application number WO2004/106377.

Humanised antibodies (which include CDR-grafted antibodies) are antibody molecules having one or more complementarity determining regions (CDRs) from a non-human species and a framework region from a human immunoglobulin molecule (see, e.g. U.S. Pat. No. 5,585,089; WO91/09967). It will be appreciated that it may only be necessary to transfer the specificity determining residues of the CDRs rather than the entire CDR (see for example, Kashmiri et al., 2005, Methods, 36, 25-34). Humanised antibodies may optionally further comprise one or more framework residues derived from the non-human species from which the CDRs were derived.

Chimeric antibodies are those antibodies encoded by immunoglobulin genes that have been genetically engineered so that the light and heavy chain genes are composed of immunoglobulin gene segments belonging to different species.

The antibodies for use in the present invention can also be generated using various phage display methods known in the art and include those disclosed by Brinkman et al. (in J. Immunol. Methods, 1995, 182: 41-50), Ames et al. (J. Immunol. Methods, 1995, 184:177-186), Kettleborough et al. (Eur. J. Immunol. 1994, 24:952-958), Persic et al. (Gene, 1997 187 9-18), Burton et al. (Advances in Immunology, 1994, 57:191-280) and WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

Fully human antibodies are those antibodies in which the variable regions and the constant regions (where present) of both the heavy and the light chains are all of human origin, or substantially identical to sequences of human origin, not necessarily from the same antibody. Examples of fully human antibodies may include antibodies produced for example by the phage display methods described above and antibodies produced by mice in which the murine immunoglobulin variable and constant region genes have been replaced by their human counterparts eg. as described in general terms in EP0546073 B1, U.S. Pat. No. 5,545,806, U.S. Pat. No. 5,569,825, U.S. Pat. No. 5,625,126, U.S. Pat. No. 5,633,425, U.S. Pat. No. 5,661,016, U.S. Pat. No. 5,770,429, EP 0438474 B1 and EPO463151 B1.

In one example the antibodies for use in the present invention may be derived from a camelid, such as a camel or llama. Camelids possess a functional class of antibodies devoid of light chains, referred to as heavy chain antibodies (Hamers et al., 1993, Nature, 363, 446-448; Muyldermans, et al., 2001, Trends. Biochem. Sci. 26, 230-235). The antigen-combining site of these heavy-chain antibodies is limited to only three hypervariable loops (H1-H3) provided by the N-terminal variable domain (VHH). The first crystal structures of VHHs revealed that the H1 and H2 loops are not restricted to the known canonical structure classes defined for conventional antibodies (Decanniere, et al., 2000, J. Mol. Biol, 300, 83-91). The H3 loops of VHHs are on average longer than those of conventional antibodies (Nguyen et al., 2001, Adv. Immunol., 79, 261-296). A large fraction of dromedary heavy chain antibodies have a preference for binding into active sites of enzymes against which they are raised (Lauwereys et al., 1998, EMBO J, 17, 3512-3520). In one case, the H3 loop was shown to protrude from the remaining paratope and insert in the active site of the hen egg white lysozyme (Desmyter et al., 1996, Nat. Struct. Biol. 3, 803-811). Accordingly, whilst clefts on protein surfaces are often avoided by conventional antibodies, heavy-chain antibodies of camelids have been demonstrated to be capable of entering enzyme active sites, largely due to the compact prolate shape of VHH formed by the H3 loop (De Genst et al., 2006, PNAS, 103, 12, 4586-4591 and WO97049805).

It has been suggested that these loops can be displayed in other scaffolds and CDR libraries produced in those scaffolds (See for example WO03050531 and WO97049805). Accordingly as detailed herein above, scaffolds containing such loops and CDRs may be used in the present invention.

In one example the antibodies for use in the present invention may be derived from a cartilaginous fish, such as a shark. Cartilaginous fish (sharks, skates, rays and chimeras) possess an atypical immunoglobulin isotype known as IgNAR. IgNAR is an H-chain homodimer that does not associate with light chain. Each H chain has one variable and five constant domains. IgNAR V domains (or V-NAR domains) carry a number of non canonical cysteines that enable classification into two closely related subtypes, I and II. Type II V regions have an additional cysteine in CDRs 1 and 3 which have been proposed to form a domain-constraining disulphide bond, akin to those observed in camelid VHH domains. The CDR3 would then adopt a more extended conformation and protrude from the antibody framework akin to the camelid VHH. Indeed, like the VHH domains described above, certain IgNAR CDR3 residues have also been demonstrated to be capable of binding in the hen egg white lysozyme active site (Stanfield et al., 2004, Science, 305, 1770-1773.

Examples of methods of producing VHH and IgNAR V domains are described in for example, Lauwereys et al, 1998, EMBO J. 1998, 17(13), 3512-20; Liu et al., 2007, BMC Biotechnol., 7, 78; Saerens et al., 2004, J. Biol. Chem., 279 (5), 51965-72.

Given the ability of certain VHH, IgNAR and other such antibody domains and structures with protruding CDRs to bind into clefts on target proteins, in one example these antibodies are the preferred antibodies for use in the present invention. Also, given the convex nature of these CDRs the binding site on the target protein is often relatively small and focused, making these useful antibodies for identifying a cluster of closely situated contact atoms suitable for use in compound fragment growth. Accordingly in one embodiment an antibody for use in the present invention is a VHH antibody or epitope binding fragment thereof, such as a VHH domain antibody or one or more CDRs derived therefrom. In one embodiment an antibody for use in the present invention is an IgNAR antibody or epitope binding fragment thereof, such as a IgNAR V domain or V-NAR domain or one or more CDRs derived therefrom. In one embodiment an antibody for use in the present invention is a CDR3 containing antibody or scaffold protein comprising a CDR3 derived from a VHH domain antibody or an IgNAR antibody. Typically such antibodies or scaffolds contain CDR3 regions which are greater than 10 amino acids in length. In one example the CDR3 regions are greater than 20 amino acids in length. In one example the CDR3 regions are up to 30 amino acids in length. In one example the CDR3 regions are between 20 and 30 amino acids in length.

The function modifying antibody molecules used in the present invention preferably have a high binding affinity for the target protein, preferably nanomolar or picomolar. In one example the neutralising antibody molecules used in the present invention preferably have a high binding affinity for the target protein, preferably nanomolar or picomolar. Affinity may be measured using any suitable method known in the art, including BIAcore using natural or recombinant target protein. In one example the antibody molecules for use in the present invention have a binding affinity of about 10 nM or better. In one example the antibody molecules for use in the present invention have a binding affinity of about 1 nM or better. In one example the antibody molecules for use in the present invention have a binding affinity of about 500 pM or better. In one example the antibody molecules for use in the present invention have a binding affinity of about 200 pM or better. In one embodiment the antibody molecules for use in the present invention have a binding affinity of about 100 pM or better. In one embodiment the antibody molecules for use in the present invention have a binding affinity of about 50 pM or better. It will be appreciated that the affinity of antibodies generated in the method of the present invention may be altered using any suitable method known in the art. Such variants can be obtained by a number of affinity maturation protocols including mutating the CDRs (Yang et al., J. Mol. Biol., 254, 392-403, 1995), chain shuffling (Marks et al., Bio/Technology, 10, 779-783, 1992), use of mutator strains of E. coli (Low et al., J. Mol. Biol., 250, 359-368, 1996), DNA shuffling (Patten et al., Curr. Opin. Biotechnol., 8, 724-733, 1997), phage display (Thompson et al., J. Mol. Biol., 256, 77-88, 1996) and sexual PCR (Crameri et al., Nature, 391, 288-291, 1998). Vaughan et al. (supra) discusses these methods of affinity maturation.

In the present invention one or more antibodies are obtained which bind to the target protein and alter the activity of the target protein in a desirable way as determined using suitable screens as described herein above. In one embodiment one antibody or fragment thereof is generated in the method of the present invention. In one embodiment two antibodies or fragments thereof which bind the target protein are produced. In one embodiment three antibodies or fragments thereof which bind the target protein are produced. In one embodiment a panel of three or more antibodies or fragments thereof which bind the target protein and alter the biological activity of the target protein are produced. It will be appreciated that such a panel of antibodies may comprise 3, 4, 5, 6, 7, 8, 9 or 10 or more antibodies. It will also be appreciated that each of the antibodies may modulate the activity of the target protein to the same or a different extent and that they may bind in the same, similar, overlapping or different locations on the target protein. Furthermore, each of the antibodies may be generated by the same or different means e.g. they may be obtained from the same or different species and/or may be the same or different types of antibodies, e.g. humanised or chimeric and/or they may be of different formats e.g. VHH or IgNAR domains. In another example more than one antibody may be derived from a single parent antibody, for example by mutagenesis, thus generating a panel of 2 or more related antibodies which bind the target protein at different locations and/or with different affinities and/or with varying abilities to modulate the activity of the target protein. Furthermore such mutagenesis may be used to generate panels of antibodies, through methods such as alanine scanning in order to help validate contact atoms on the antibody and/or to prioritise pharmacophore sites by allowing critical residues/contact atoms on both the antibody and the target protein to be identified. Accordingly, in one embodiment at least one of the antibodies obtained in step (a) of the method is generated by mutagenesis of another antibody obtained in step (a) of the method.

### Antibody:Target 3D Structural Representation

In the present invention one or more antibodies are obtained which bind to the target protein and alter the biological activity of the target protein in a desirable way (e.g. inhibit or stimulate activity) as determined using suitable screens as described herein. A three dimensional structural representation of at least one such antibody in complex with the target protein is subsequently generated in order to obtain information about where the antibody is binding the target protein and which amino acid residues and hence atoms on the target protein and the antibody are in contact with each other or interact with each other. Preferably, where more than one antibody has been obtained which binds the target protein and modulates its activity, structural information is obtained for each of the antibodies in complex with the target protein. It will be appreciated that steps (a) and (b) may be iterative and that the 3D structure of each antibody in association with the target protein may be generated before another antibody is obtained.

Any suitable method known in the art can be used to generate the three dimensional structural representation of an antibody:target protein complex. Examples of such methods include NMR and X-ray crystallography. Preferably X-ray crystallography is used. As set out herein above, the target protein may be the mature protein or a suitable fragment or derivative thereof.

X-ray crystals will typically comprise crystallized polypeptide complexes corresponding to wild-type or mutated target protein in complex with an antibody or antibody fragment wherein the antibody fragment is for example an antibody VHH domain, IgNAR domain, dAb, Fab or Fab′ fragment as described herein above. Such crystals include native crystals, in which the crystallized protein:antibody fragment complex is substantially pure and poly-crystals, in which the crystallised protein:antibody fragment complex is in association with one or more additional compounds, including but not limited to, inhibitors, antagonists, antibodies, or one or more receptor(s).

Preferably the crystals produced are of sufficient quality to permit the determination of the three dimensional X-ray diffraction structure of the crystalline polypeptide(s) to high resolution, preferably to a resolution of greater than about 3 Å, typically in the range of about 1 Å to about 3 Å. Generally crystals are grown by dissolving substantially pure polypeptide complexes in an aqueous buffer that includes a precipitant at a concentration just below that necessary to precipitate the polypeptide complexes. Water is then removed by controlled evaporation to produce precipitating conditions which are maintained until crystal growth ceases. Poly-crystals are prepared by soaking a native crystal prepared according to the above method in a liquor comprising the compound to be added to the poly-crystal of the desired complex. Alternatively, the poly-crystals may be prepared by co-crystallising the polypeptide complexes in the presence of the compound according to the method discussed above.

The three dimensional structural information obtained will typically include the atomic structure coordinates of the crystallised polypeptide complex or poly-complex, or the atomic structure coordinates of a portion thereof, such as, for example, the antibody binding site or a neutralising epitope, but may include other structural information, such as vector representations of the atomic structures coordinates etc.

Preferably the three-dimensional structural information regarding the antibody-target protein complex comprises the atomic structure co-ordinates. Preferably the structural information comprises all or part of a binding site on the target protein bound by the antibody.

In the present invention the three-dimensional structural information is used to generate a three-dimensional representation of the antibody in complex with the target protein using suitable models and computer programmes known in the art (See for example, CCP4 Collaborative Project Number 4 (1994), The CCP4 Suite; Programs for Protein Crystallography, Acta Cryst, D50, 760-763). This 3D representation is then used to identify one or more contact residues or atoms on the target protein and the antibody that fall within the antibody binding site or sites on the target protein. Preferably each atom on the target protein and each atom on the antibody that are in contact with or interact with each other in the antibody binding site are identified, such that antibody-target protein pairs of atoms are identified. Where the structure of more than one antibody-target protein complex is generated then 1 or more atoms, preferably 1 or more pairs of atoms, in the binding site of each antibody is/are identified. Typically each pair of atoms in the binding site of each antibody are identified.

Typically an antibody binding site of the present invention comprises one or more of the residues or atoms on the target protein that are in contact with or interact with an antibody identified in the method of the present invention and one or more residues or atoms on the antibody that make those contacts or interactions with the target protein.

In one example the contact atoms on the target protein which fall within a binding site of the antibody are those that are in contact with or interact with any part of the antibody, including the variable and constant regions and including any side chain and backbone atoms within those regions. In one example the contact atoms on the target protein in a binding site are those that are in contact with or interact with the variable regions of the antibody. In one example the contact atoms on the target protein in a binding site are those that are in contact with or interact with any one of the CDRs and any part of the variable region frameworks, including side chain and backbone atoms within the CDRs and framework regions. In one example the contact atoms on the target protein that fall within the binding site are those which are in contact with or interact with any part of one or more of framework 1, 2, 3 or 4 of the light chain. In one example the contact atoms on the target protein that fall within the binding site are those which are in contact with or interact with any part of one or more of framework 1, 2, 3 or 4 of the heavy chain. In one example the contact atoms on the target protein in a binding site are those which are in contact with or interact with any part of one or more of the CDRs of the antibody. In one example the contact atoms on the target protein that fall within the binding site are those which are in contact with or interact with any part of one or more of CDRH1, CDRH2, CDRH3, CDRL1, CDRL2 or CDRL3 of the antibody. In one example the contact atoms on the target protein that fall within the binding site are those which are in contact with or interact with any part of CDRH1 and/or CDRH2 and/or CDRH3 of the antibody. In one example the contact atoms on the target protein in a binding site are those which are in contact with or interact with any part of CDR3 of the heavy chain of the antibody. In one example the contact atoms on the target protein in a binding site are those which are in contact with the CDRH3 of the antibody, for example the CDR3 of a VHH domain.

In one example, the contact atoms on an antibody that fall within a binding site of the antibody are those contact atoms that interact with or are in contact with any part of the target protein including any side chain and backbone atoms on the target protein. Accordingly the contact atoms on the antibody that fall within a binding site are typically within the variable and/or constant regions, including any side chain and backbone atoms within those regions. In one example the contact atoms on an antibody in a binding site are those within the variable regions of the antibody that are in contact with or interact with the target protein. In one example the contact atoms on the antibody that fall within the binding site are those which are in any part of one or more of framework 1, 2, 3 or 4 of the light chain of the antibody which make contact with or interact with the target protein. In one example the contact atoms on the antibody that fall within the binding site are those which are in any part of one or more of framework 1, 2, 3 or 4 of the heavy chain of the antibody which make contact with or interact with the target protein. In one example the contact atoms on the antibody are those within any one of the CDRs and any part of the variable region frameworks, including side chain and backbone atoms within the CDRs and framework regions. In one example the contact atoms on the antibody that fall within the binding site are those which are in any part of one or more of the CDRs of the antibody which make contact with or interact with the target protein. In one example the contact atoms on the antibody that fall within the binding site are those which are in any part of one or more of CDRH1, CDRH2, CDRH3, CDRL1, CDRL2 or CDRL3 of the antibody which make contact with or interact with the target protein. In one example the contact atoms on the antibody that fall within the binding site are those which are in any part of CDRH1 and/or CDRH2 and/or CDRH3 of the antibody which make contact with or interact with the target protein. In one example the contact atoms on an antibody that fall within the binding site are those which are in any part of CDR3 of the heavy chain of the antibody which make contact with or interact with the target protein. In one example the contact atoms on the antibody which fall within a binding site of the antibody are those which are in within the CDRH3 of the antibody, for example the CDR3 of a VHH domain.

It will be appreciated that there may be more than one binding site or cluster of residues or atoms on a target protein in contact or interacting with an antibody, for example where there is more than one interaction between different parts of the antibody and the target protein. Accordingly, in one example, once the 3D structural representation of each antibody-target protein complex has been generated the antibody binding site can be determined and selected by visual inspection. For example suitable antibody binding sites may be identified by structural features of the target protein and/or the topography of the target protein and/or whether these sites are considered druggable. For example where the antibody binds into a naturally occurring groove or cleft on the target protein or where such a groove or cleft is generated as a result of antibody binding such a site may be preferred. Indeed these sites may already be known sites for e.g. receptor or ligand binding. Alternatively, previously unknown function modifying sites may be revealed for the first time by antibody binding and these sites may be preferred. In one example the binding site may be considered to be the site in contact with the CDRH3 of the antibody, for example the CDRH3 of a VHH antibody. It will be appreciated that more than one site may be selected if appropriate.

It will also be appreciated that where more than one antibody has been obtained which binds the target protein that the collection of structural information provided from the binding sites of each of those antibodies may be ‘pooled’ to help select one or more antibody binding sites. For example where the function modifying antibodies frequently bind in a certain location that binding site may be preferentially selected. Where more than one antibody-target protein complex 3D structure has been determined this information may be combined to aid the selection of the antibody binding site and hence the atoms on the target protein and/or antibody that may be used in the present invention. For example where the majority of antibodies bind in a particular region of the target protein, frequently occurring interactions within that region may be preferentially selected. Accordingly pairs of contact atoms from more than one antibody may be identified in step (b) for example, a contact atom pair from the CDRH2 of a first antibody and a contact atom pair from the CDRH3 of a second antibody and such contact atom pairs used to guide the growth of a compound fragment in step (f).

In one example at least one contact atom on the target protein in an antibody binding site is identified. In one example at least one contact atom on the antibody that falls within an antibody binding site is identified. In one example at least one contact atom on the target protein and the contact atom on the antibody that interacts with it is identified i.e. at least one pair of atoms are identified. The intermolecular interactions between antibody and target protein atom are typically electrostatic interactions such as hydrogen bonds and van der Waals non-polar interactions. Preferably all the antibody-target protein contact atom pairs within an antibody binding site are identified.

Where more than one contact atom pair is identified in a given binding site e.g. one on the target protein and one on the antibody, the protein contact atoms are preferably within a suitable distance of one another to be useful in subsequent fragment growth, as described herein below. In one example the contact atoms identified in the binding site will ideally be within a suitable distance of one another, typically about 1 Å to about 30 Å based on the shortest non-covalent atomic interaction or hydrogen bond distance and the longest distance between protein atoms that can be considered within the same binding site. The identified protein contact atoms can be prioritised through experimental protein mutagenesis studies or computational methods such as molecular mechanics free energy calculations [Moreira et al. J Comput Chem. 2007 February; 28(3):644-54].

### Compound Fragment Screening

In step (c) of the method of the present invention one or more compound fragments that bind the target protein are obtained.

Compound fragments for use in the present invention typically have a molecular weight of less than 600 Da. In one example the compound fragments have a molecular weight of less than 500 Da. In one example the compound fragments have a molecular weight of less than 400 Da. In one example the compound fragments have a molecular weight of less than 350 Da. In one example the compound fragments have a molecular weight of less than 300 Da. In one example the compound fragments have a molecular weight of less than 250 Da. In one example the compound fragments have a molecular weight of less than 200 Da. Such fragments are typically small, simple compounds, usually consisting of no more than one or two rings with a few substituents.

By screening libraries of such compound fragments it is usually possible to identify small compound fragments that bind very efficiently to the target protein, albeit only through a low number of interactions with the target protein, hence these are usually low affinity interactions. These compound fragment hits can be considered building blocks that can be combined e.g. merged or linked, to form larger and potentially much more potent and druglike lead compounds. Alternatively, these hits can be ‘seeds’ or ‘anchor points’ which can be synthetically expanded into lead compounds, picking up increasingly more interactions with the target protein. In one example a mixture of both approaches may be used to generate subsequent compounds for screening.

Typically fragment-based screening involves screening a number of compound fragments, typically several thousand compounds, to find low-affinity fragments with Kd values in the high micromolar to millimolar range. For a review of fragment based screening methods see Hajduk and Greer, 2007, Nat. Rev. Drug. Discov. 6(3), 211-219.

Suitable compound fragment libraries may be designed using any suitable methods known in the art, whereby selection of compound fragments for inclusion in the libraries may be based on the presence or absence of desirable or undesirable chemical functionality and other constraints may be placed on the library such as solubility, shape, flexibility or spectral properties. Additionally, the strategy for subsequent chemistry on the fragments can also influence the design of the library. A review of fragment library strategies is provided in Baurin et al., 2004, J. Chem. Inf. Comput. Sci, 44, 2157-2166; Hubbard et al., 2007, Curr. Opin. Drug Discov. Devel., 10, 289-297; Zartier and Shapiro, 2005, Curr. Opin. Chem. Biol., 9, 366-370.

In the method of the present invention compound fragments are screened for binding to the target protein, either directly or virtually. The binding information can be obtained using any suitable method known in the art. For example an overview of the different approaches is given in the book ‘Fragment-based approaches in drug discovery’ (Jahnke, Erlanson, Mannhold, Kubinyi & Folkers (2006), published by Wiley) and also the review of Rees and coworkers (Rees et al. (2004) Nature Rev. Drug Discov. 3, 660-672).

Experimental methods useful for determining binding of compound fragments to proteins include but are not limited to:

Protein X-ray crystallography [Hartshorn et al. (2005) J. Med. Chem. 48, 403-41 3]. Efficient fragment screening using protein X-ray crystallography requires the soaking of cocktails of fragments into pre-formed crystals of a target protein. After collection of the X-ray data, the identification of the fragments from the cocktail is reliant on manual or automated analysis of the resultant electron density. The outcome of these studies is information regarding which fragments bind to the protein target and the actual binding configuration in the active site. No information is obtained on the actual binding strength or affinity.
NMR-based screening [Shuker et al. (1996) Science 21A, 1531-1534], or Structure-Activity-Relationship (SAR) by NMR, involves identifying and interpretation of the chemical shifts in the NMR spectrum as a result of the fragment to a target protein of interest. The result is information regarding the fragments that bind to the protein target. Typically, no information is obtained of the actual binding strength or affinity. Target Immobilized NMR screening may also be used (Vanwetswinkel et al., 2005, Chemistry & Biology, 12(2): 207-216).
The use of disulfide bonds to stabilize the binding of a fragment to the target protein [DeLano (2002) Curr. Opin. Struct. Biol. 12, 14-20]. This is achieved by placing a sulfur-containing amino acid called a cysteine on the surface of the protein and to screen the protein against a collection of sulfur-containing fragments. Fragments that bind near the cysteine form disulfide bonds with the protein, increasing the weight of the protein and allow the detection of the fragments by mass spectrometry. The outcome is a list of fragments that bind to the protein. No particular information is obtained regarding the fragments binding strength.
Microcalorimetry-based fragment screening has been described in an application note of MicroCal LLC (USA) [‘Divided we fall? Studying low affinity real molecular species of ligands by ITC’, MicroCal LLC, USA, 2005] in which the heat generated by the fragment-protein binding process is measured and converted in thermodynamical parameters such as entropy and enthalpy measures. The outcomes of the experiments are the identities of the binding fragments and optionally the corresponding binding affinities.
In-vitro binding assays which have been adapted to measure the binding of low affinity fragments have also been described [Boehm et al. (2000) J. Med. Chem. 43, 2664-2674]. The results of these experiments are a set of fragments with their corresponding binding affinities for a particular protein target.
Sedimentation analysis is a novel technology that has been described to measure fragment/protein interactions [Lebowitz et al. (2002) Protein Sci. 11, 2067-2079]. Sedimentation equilibrium measures the concentrations of the components at equilibrium in solution, and the readout from an sedimentation equilibrium experiments is an absorbance versus distance curve. The outcomes of the experiments are the identities of the fragments that show binding affinity for a particular protein target.
Solid-phase detection is a general term covering a wide range of technologies that share a common working principle in which both a bioreceptor and a signal transducer are combined to detect the binding of fragments to proteins. The best known solid-phase detection method is surface plasmon resonance (SPR), which has originally been described and implemented by Graffinity Pharmaceuticals GmbH (Germany). The process involves a highly parallel production of chemical microarrays using proprietary, highly defined surface chemistry, followed by the simultaneous detection of protein interactions to 10,000 fragments via SPR imaging. Interaction data are combined with physicochemical compound data to interpret the array results. One specific example of SPR is BIAcore™, which has been used for screening fragment libraries (Metz et al., 2003, Meth.Principles.Med. Chem., 19, 213-236 and Neumann et al., 2005, Lett. Drug. Des. Discovery, 2, 590-594). Alternative solid-phase detection methods include but are not limited to the rupture event scanning (REVS) and resonant acoustic profiling (RAP) technologies commercialized by Akubio Ltd (UK), reflectance interference (RIf), total internal reflection fluorescence (TIRF), and the microcantelever technology as commercialized by Concentris GmbH (Suisse).
Capillary electrophoresis has also been mentioned as a tool to measure fragment/protein interaction [Carbeck et al. (1998) Ace.Chem. Res. 31, 343-350].

It will be appreciated that one or more of the methods described above may be used. For example NMR may be used to determine fragment binding and this may combined with BIAcore™ screening to determine affinities and ‘rank’ the fragment hits identified. In one example BIAcore™ is used to simultaneously determine binding to the target protein and affinity.

Complementary to the experimental approaches mentioned above to generate fragment-binding data, information gathered from literature sources may also be useful in generating knowledge about the affinity of certain fragments to specific target proteins.

Whatever approach is used to collect the fragment binding data, the result is a list of compound fragment structures which are known, or believed, to bind to the target protein. Quantitative affinity information in the form of dissociation or IC50 values is useful, but not essential.

It will be appreciated that where suitable structural information regarding the target protein is already available, such as publicly available data, virtual screening methods may be used to identify fragments that are expected to bind to the target protein. Virtual screening methods useful for determining binding of compound fragments to proteins include but are not limited to: GLIDE [Friesner et al J Med. Chem. 2004 Mar. 25; 47(7):1739-49] and GOLD [Jones et al. J Mol. Biol. 1997 Apr. 4; 267(3):727-48]. In one example the protein contact atoms, previously described, may identify pharmacophoric features which can be used as computational parameters in the structure-based virtual screen.

In one example the structure of the target protein crystal may be probed with a variety of different chemical fragments to determine optimal sites for interactions between such candidate molecules and the target protein. Small molecule fragments that would bind tightly to those sites can then be designed, and optionally synthesised and tested for their ability to bind to the target protein.

In another example computational screening of small molecule databases is used to identify chemical fragments that can bind in whole, or in part to the target protein. This screening method and its utility are well known in the art. For example, such computer modelling techniques have been described in WO 97/16177 and WO2007/011392.

In one embodiment the computational screen further comprises the steps of synthesising the candidate fragment and screening the candidate fragment for the ability to bind the target protein as described herein above.

Typically one or more compound fragments which bind the target protein are identified in step (c) of the method of the present invention. In one example two or more fragments are identified. In one example three or more fragments are identified. In one example four or more fragments are identified. In one example five or more fragments are identified. In one example ten or more fragments are identified. In one example twenty or more fragments are identified. In one example fifty or more fragments are identified.

It will be appreciated that not all fragments identified may be selected for further 3D structural analysis, for example if a large number of fragments are identified. Certain fragments may be selected based on their synthetic tractability and/or likelihood of compliance with Lipinski's rule of five (Lipinski et al., 1997, Adv. Drug.Del.Rev, 23, 3-25).

Alternatively, or in addition, where the affinity of the fragments for the target protein has been determined this may be used to rank the fragments, such that those fragments with the highest affinity or ligand efficiency go on for further testing. Ligand efficiency is binding affinity normalised by size (i.e. potency/size)

Alternatively or in addition, analogues of the compound fragments known to bind the target protein may be identified and selected based on for example, anticipated improved binding or synthetic tractability. It will be appreciated that these analogues could be tested for binding to the target protein either directly or virtually.

Alternatively or in addition compound fragments may be ranked based on competition assays conducted using one or more of the antibodies identified as described herein above, such that those fragments which bind in the same region as one or more of the antibodies identified are selected e.g. those compound fragments that cannot bind the target protein in the presence of the antibody are preferentially selected.

It may be that a combination of the different factors described above may be used to select one or more compound fragments for using 3D structural analysis.

3D Structural Analysis of Compound Fragments

In step (d) of the method of the present invention a three dimensional structural representation is generated for one or more of the selected compound fragments in combination with the target protein. Suitable methods for such structural representations include X-ray crystallography and NMR. Preferably X-ray crystallography is used.

The three-dimensional structural representation generated provides information about how and where the compound fragment is binding the target protein. This information can then be used to compare with the 3D structural information obtained for the binding of one or more function modifying antibodies to the target protein. The key interactions identified using molecular modelling programs such as Sybyl [www.tripos.com] may be used to generate a pharmacophore for structure based drug design.

One or more compound fragments that bind the target protein within or in the same vicinity as an antibody binding site identified by the method of the present invention are selected in step (e) for use in the synthesis of one or more candidate small molecule compounds, for example by compound fragment growth.

In one example a compound fragment will be selected if it makes an intermolecular interaction with one or more protein contact atoms on the target protein and/or the antibody identified as falling within an antibody binding site. An intermolecular interaction would include an electrostatic interaction such as a hydrogen bond or lipophillic van der Waals interactions.

In one example a compound fragment will be selected if it makes an intermolecular interaction in the vicinity of an antibody binding site. An intermolecular interaction would include an electrostatic interaction such as a hydrogen bond or lipophillic van der Waals interactions. In one example a compound fragment will be selected if it makes an intermolecular interaction in the vicinity of one or more protein contact atoms on the target protein and/or the antibody identified as falling within an antibody binding site in step (b). The term ‘in the vicinity of’ as used herein refers to a distance of 1-30 Å. In one example the term ‘in the vicinity of’ refers to a distance of 1-20 Å or 1-10 Å. In one example a compound fragment will be selected if it makes an intermolecular interaction within 1-30 Å of one or more protein contact atoms on the target protein and/or the antibody identified as falling within an antibody binding site in step (b).

As set out above, selecting which antibody binding site should be targeted by a compound fragment and hence which fragment may be selected may be based on the collective data of more than one antibody or for example antibody binding site data and known ligand or receptor binding sites. Where more than one antibody which binds in the same region of the target protein has been identified the contact atoms identified on the target protein which interact with the antibodies can be combined for use in the growth of fragments.

For example, the frequency of antibodies binding in a given region of the target protein may be used to guide fragment growth or analoguing into a particular area of chemical space or 3D location to coincide with the location of antibody residues or atoms which interact with residues or atoms on the target protein. Accordingly, the combined antibody-target protein contact information obtained may be used to direct compound fragment growth to a particular region of the target protein and/or to aid selection of a compound fragment that binds in that region and/or may provide a composite collection of residues within a particular region of the target protein to guide compound fragment growth and/or may direct compound fragment growth to a certain area of chemical space or 3D location.

It will be appreciated that steps (c) and (d) may be iterative and that the 3D structure a compound fragment in association with the target protein may be generated before another compound fragment is obtained. In addition steps (c) and (d) may be repeated until a compound fragment that binds within or in the vicinity of the antibody binding site identified in step (b) is identified.

### Compound Fragment Growth

Once a compound fragment binding within or in the vicinity of an antibody binding site has been identified, the contact atom information obtained from a given antibody binding site or sites can be used to guide the growth of the compound fragment to generate a candidate small molecule compound or a library of such compounds.

Typically the goal of such target guided synthesis is to build up molecules in a modular way to produce assembled molecules which have a higher binding affinity for the target protein than their individual parts or fragments and are able to exert a desired biological effect on the target protein. It will be appreciated that other structural information e.g. from ligands, inhibitors, other compound fragments may be used in this process, in combination with the antibody contact information.

Using the structural information obtained from the compound fragment(s)-target protein interaction and the antibody-target protein interaction any suitable fragment assembly/growth/rational method may be used to grow the fragment(s) selected to generate novel small molecule candidate compounds. Such methods include but are not limited to SAR by NMR, dynamic libraries, analoguing and virtual screening.

Methods of fragment based lead generation and subsequent rational design of more potent hit analogues and small molecules are known in the art, see for example Szczepankiewicz et al., Journal of the American Chemical Society (2003), 125(14), 4087-4096; Raimundo et al., Journal of Medicinal Chemistry (2004), 47(12), 3111-3130, Braisted et al., Journal of the American Chemical Society (2003), 125(13), 3714-3715, Huth et al., Chemical Biology & Drug Design (2007), 70(1), 1-12, Petros et al., Journal of Medicinal Chemistry (2006), 49(2), 656-663, Geschwindner et al., Journal of Medicinal Chemistry (2007), 50(24), 5903-5911, Edwards et al., Journal of Medicinal Chemistry (2007), 50(24), 5912-5925 and Hubbard, et al., Current Topics in Medicinal Chemistry (2007), 7(16), 1568-1581.

Typically such methods of compound fragment growth include iterative structure-based drug design to optimise a lead compound. Methods also include ‘analoguing’ of the compound fragments to identify nearest neighbours or similar compound fragments to those identified by screening and these may then be screened for binding to the target protein.

Growing the fragment(s) identified can be performed by adding functionality that binds to additional sub-sites on the protein surface. This can be achieved by searching a database of available chemicals for compounds containing the same sub-structures as the fragment or by synthesizing small libraries that add functionality to key attachment points on the fragment. The position and orientation of binding of the fragment to the target protein and the structural information obtained from the antibody which binds in the same vicinity can be used to guide the computational search or library synthesis. It will be appreciated that where additional relevant structural information is available, such as ligand binding, this may also be used to guide fragment growth.

Accordingly, ‘growing’ the compound fragment may encompass a number of activities including adding functionality, making small libraries, searching databases, computational searching and/or ‘analoguing’.

Typically ‘growing’ a compound fragment in the method of the present invention involves directing chemical synthesis to:

(1) interact with a contact atom on the target protein identified as falling within an antibody binding site
and/or
(2) occupy the same or similar chemical space or 3D location as an antibody contact atom within the antibody binding site
In one example, step (f) of the method comprises growing the compound fragment selected in step (e) to generate one or more candidate compounds which interact with one or more contact atoms on the target protein identified in step (b) by directing the chemical growth of the compound fragment such that the expanded fragment occupies the same or similar chemical space or 3D location as one or more contact atoms on the antibody identified in step (b). Preferably each contact atom on the antibody is the corresponding contact atom from the antibody-target protein atom pair identified in step (b).

Where fragment growth is directed to occupy the same or similar chemical space or 3D location as an antibody contact atom within the antibody binding site the fragment may incorporate the same intermolecular interactions or molecular shape as used by the antibody. This may include for example electrostatic interactions, hydrogen bonds, lipophillic or van der Waals interactions.

Thus by using the spatial location and the type of intermolecular interactions of the antibody contact atoms the fragment can be grown to mimic the antibody interaction with the contact atoms on the target protein. This spatial location is termed herein the 3D location or chemical space. The same chemical space or 3D location can therefore be identified in a 3D representation e.g. X ray crystallography. It will be appreciated that it does not have to be exactly the same as the antibody atom location, particularly if a different atom is used, but will be largely equivalent and may occupy similar chemical space.

Where fragment growth is directed to interact with atoms on the target protein without incorporating atoms which occupy the same 3D location or chemical space as the antibody, the nature of the antibody atoms and their interaction with the target protein may still be used to grow the fragment to another suitable location which makes an equivalent interaction, e.g. by using virtual screening tools and/or modelling tools.

As set out above where more than one antibody which binds in the same region of the target protein has been identified the contact atoms identified can be combined for use in the growth of fragments. This information may be used to incorporate one or more new interactions into the fragments at once or they may be generated iteratively. It will be appreciated that steps (f) to (h) may be iterative such that the compound selected in step (e) may be grown in stages to interact with further contact atoms identified in step (b) from the same or different antibodies. Similarly step (i) encompasses this concept of iterative further improvements to the candidate compounds selected in step (h).

A series of new compounds may be generated and these may then be screened for improved affinity for the target protein and/or for improved binding to the target protein and/or improved potency and/or the ability to alter the biological activity of the target protein. It will be appreciated that this may involve iterative rounds of further chemistry and fragment growth, optionally incorporating one or more further interactions with contact atoms on the target protein identified from the antibody binding structural representations.

Accordingly in one embodiment the present invention provides a method of generating a candidate compound or library of candidate compounds that may be used to generate a small molecule compound that alters the activity of a target protein.

The candidate small molecule compound or compounds generated using these methods can then be tested for their ability to alter the biological activity of the target protein using suitable screening methods as described herein. Such small molecule compounds are typically <650 Da in size and are not peptide mimics.

The one or more candidate compounds generated in step (f) of the method are tested in step (g) for improved affinity for the target protein and/or improved potency and/or improved ligand efficiency and/or ability to alter the biological activity of the target protein. As set out above growth of the compound fragments may be iterative and hence steps (f) and (g) may be repeated more than once. A candidate compound produced in step (f) and tested in step (g) therefore has the potential to be a small molecule compound that alters the activity of the target protein without requiring further alteration to it's chemical structure to be used as a drug. Alternatively a candidate compound produced in step (f) and tested in step (g) may require further modification in order to generate a small molecule compound e.g. to improve the extent to which it modulates the activity of the target protein and/or to make the compound more drug-like. A compound selected in step (h) therefore may be a small molecule compound, as described herein below, which modulates the activity of the target protein to the required extent for use as a drug. Alternatively a compound selected in step (h) may be a candidate compound requiring further chemistry and screening in order to generate a suitable small molecule compound. Accordingly in step (i) of the method of the present invention further chemistry is optionally performed on a compound selected in step (h), optionally by repeating steps (f) and (g) to grow the fragment selected in (h) to interact with one or more further contact atoms identified in step (b) from the same or different antibodies.

A small molecule compound according to the present invention is typically less than 650 Da in size and is not a peptide. In one example a small molecule compound of the present invention has a molecular weight of less than 600 Da. In one example a small molecule compound of the present invention has a molecular weight of less than 550 Da. In one example a small molecule compound of the present invention has a molecular weight of less than 500 Da.

In one example a small molecule produced by the method of the present invention complies with at least one, preferably all of Lipinski's rule of five (Lipinski et al., 1997, Adv. Drug. Del. Rev, 23, 3-25).

Accordingly in one example a small molecule produced by the method of the present invention contains no more than five hydrogen bond donors (nitrogen or oxygen atoms with one or more hydrogen atoms).

In one example a small molecule produced by the method of the present invention contains no more than ten hydrogen bond acceptors (nitrogen or oxygen atoms).

In one example a small molecule produced by the method of the present invention has an octanol-water partition coefficient log P of less than 5.

In one example a small molecule produced by the method of the present invention has no more than two violations of the following criteria:
contains no more than five hydrogen bond donors (nitrogen or oxygen atoms with one or more hydrogen atoms)
contains no more than ten hydrogen bond acceptors (nitrogen or oxygen atoms)
has an octanol-water partition coefficient log P of less than 5.
A molecular weight of less than 500 daltons

### Compositions/Therapeutic Uses

The compounds identified by the method of the present invention may be useful in the treatment and/or prophylaxis of a pathological condition, the present invention also provides a pharmaceutical or diagnostic composition comprising a compound of the present invention in combination with one or more of a pharmaceutically acceptable excipient, diluent or carrier. Accordingly, provided is the use of a compound of the invention for the manufacture of a medicament. The composition will usually be supplied as part of a sterile, pharmaceutical composition that will normally include a pharmaceutically acceptable carrier. A pharmaceutical composition of the present invention may additionally comprise a pharmaceutically-acceptable adjuvant.

The present invention also provides the compound produced by the method of the present invention for use in the treatment or prophylaxis of a pathological disorder that is mediated by the target protein or associated with an increased level of the target protein.

### EXAMPLES

The present invention will now be described by way of example only, in which reference is made to:

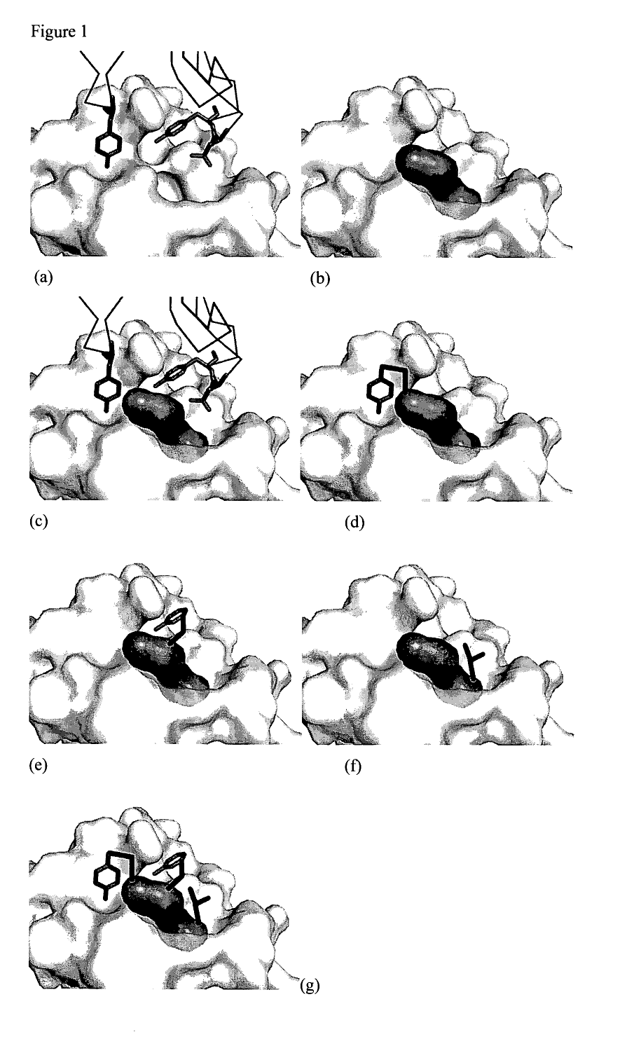

FIG. 1a. Antibody Fab fragment (black backbone) with side chain residues Tyr 1, Tyr2 and Asn 1 (dark grey ball and stick) and the target protein (white molecular surface)

FIG. 1b. NCE fragment (dark grey molecular surface) and target protein (white molecular surface)

FIG. 1c. NCE fragment (dark grey molecular surface), target protein (white molecular surface) and antibody Fab fragment (black backbone)

FIG. 1d. NCE fragment has been grown to incorporate a phenol group as defined by Tyr1

FIG. 1e. NCE fragment has been grown to incorporate a phenol group as defined by Tyr2

FIG. 1f. NCE fragment has been grown to incorporate the oxygen and nitrogen atoms as defined by the Asn1 side chain.

FIG. 1g. NCE fragment has been grown to incorporate a phenol group as defined by Tyr1, a phenol group as defined by Tyr2 and the oxygen and nitrogen atoms as defined by the Asn1 side chain.

### EXAMPLE 1

### Structure of Antibody:Target Complex

The target protein (a cytokine) was over-expressed in bacterial expression systems using standard protocols (described, for example, in Baneyx [1999] “Recombinant protein expression in Escherichia coli.” Current Opinion in Biotechnology 10, 411-421). Briefly, vector DNA encoding the target gene was transformed in Escherichia coli bacteria (Origami strain; Novagen, San Diego/Calif.). Protein expression was induced by the addition of isopropyl β-D-thiogalactopyranoside and cells were harvested after 16-20 h post induction. The target protein was extracted by resuspending the cells and passing them through a basic Z cell disruptor (Constant systems). The lysate was then cleared by centrifugation and 2-4 ml of Ni-NTA superflow beads (QIAGEN) were added to the lysate and the sample was stirred for 2 hours at 4° C. The Ni-NTA beads were subsequently washed, and the protein eluted in a buffer containing 250-500 mM imidazole. The eluted material was further purified by gel filtration in a suitable protein buffer, such as 50 mM TRIS-HCl (pH8.0), 100 mM NaCl.

An antibody which binds the target cytokine was isolated using the methods described in Babcook, J. et al., 1996, Proc. Natl. Acad. Sci. USA 93(15):7843-78481. The antibody binds the target cytokine with pM affinity and is able to neutralize the biological activity of the cytokine. A Fab fragment of the antibody was cloned expressed and purified for use in crystallography.

The antibody Fab fragment was expressed in transient mammalian cell cultures using standard protocols (described, for example, in Aricescu et al. [2006] “Eukaryotic Expression: Developments for Structural Proteomics” Acta Crystallographica D62:1114-1124). Briefly, human embryonal kidney (HEK293) cells were transfected with vector DNA encoding the heavy and light chain of the antibody. The vector contained a targeting sequence, such that expressed heavy and light chains were secreted into the medium. After 6 days, cells were separated from the medium by centrifugation and the protein was purified from the medium by using a combination of affinity chromatography (KappaSelect resin; GE healthcare, Amersham/UK) and gel filtration.

Antibody:target complex was generated by adding stoichiometric quantities of the antibody to the target protein, followed by a 2 h incubation at 4° C. and subsequently purification using gel filtration. Purified antibody:target complex was concentrated to a level appropriate for crystallization (in this case 8 mg/mL). Sitting-drop crystallization trials were set up using a Mosquito robotic dispensing system (TTP Labtech, Cambridge/UK). For the target protein described here, diffraction quality crystals grew within 5-10 days in 800 nl sitting drops containing 4 mg/ml purified antibody:target complex, 100 mM Ammonium sulphate, 50 mM MES buffer (pH 6.5), 11% (w/v) PEG-8000, equilibrated against a reservoir solution of 200 mM Ammonium sulphate, 100 mM MES buffer (pH 6.5), 22% (w/v) PEG-8000. Crystals were cryoprotected in a solution containing 80% reservoir and 20% glycerol, and flash-frozen in liquid nitrogen at 100K.

Diffraction data were collected at Diamond Light Source (Didcot/UK) beamline 102, and processed using the computer program MOSFLM (Leslie [1999] “Integration of macromolecular diffraction data” Acta Crystallographica D55:1696-1702). The crystal structure of the antibody:target complex was solved by molecular replacement using the known structures of the target protein and the antibody as search models for the program PHASER (McCoy et al. [2007] “Phaser crystallographic software” Journal of Applied Crystallography 40:658-674). The molecular replacement solution was rebuilt using the program COOT (Emsley and Cowtan [2004] “Coot: model-building tools for molecular graphics” Acta Crystallographica D60:2126-2132), and refined using the program CCP4 programs (Collaborative Project Number 4 [1994] “The CCP4 Suite: Programs for Protein Crystallography” Acta Crystallographica D50:760-763).

Analysis of the crystal structure revealed where the antibody was binding the target protein and pairs of contact atoms within the antibody binding site were identified i.e. atoms on the target protein and the antibody within the antibody binding site.

### Structure of the Target:Fragment Complex

Target protein was expressed, purified and crystallized as described above.

An NCE fragment was obtained by screening a library of small molecule fragments for specific binding to the target cytokine in a Biacore A100-based screen. The target cytokine was coupled to the chip surface, with the NCE fragments binding from the solution phase. Binding of the fragments was weak enough for no regeneration step to be necessary. A typical square wave profile of binding was obtained for this specific fragment in this assay format. The fragment identified had a binding affinity of 400 μM.

The target protein was crystallized in the apo-form and crystals were subsequently soaked in a buffer solution containing the fragment. Diffraction data were collected at the Swiss Light Source (Villingen, Switzerland) and processed using the program XDS and XSCALE (Kabsch [1993] “Automatic processing of rotation diffraction data from crystals of initially unknown symmetry and cell constants” Journal of Applied Crystallography 26:795-800). The structure of the target:fragment complex was solved by molecular replacement using the known structure of the target protein as a search model. The molecular replacement solution was rebuilt using the program COOT, and refined using the program CCP4 programs.

### Method Illustrating Antibody-Guided Fragment Growth

In the current example, a first crystal structure of a complex of a Fab fragment of a high affinity, neutralising antibody and a target cytokine was obtained, and a second crystal structure of a small molecule fragment bound to the same target cytokine in the same vicinity as the antibody binding site was obtained.

The antibody was generated according to the Selected Lymphocyte Antibody Method, and screened by Biacore to establish the affinity of the interaction with its target, and in a bioassay to establish function modifying properties. The antibody bound the target cytokine with pM affinity and was able to neutralize the biological activity of the cytokine. A Fab fragment of the antibody was cloned, expressed and purified to aid the crystallography.

The NCE fragment was obtained from a library of small molecule fragments, with specific binding to the target cytokine demonstrated in a Biacore A100-based screen. The target cytokine was coupled to the chip surface, with the NCE fragments binding from the solution phase. Binding of the fragments was weak enough for no regeneration step to be necessary. A typical square wave profile of binding was obtained for this specific fragment in this assay format. The fragment had an affinity for the target cytokine of 400 μM and a molecular weight of 205.

As expected, the antibody uses a number of additional atoms to contact the antigen, compared to the fragment, allowing the opportunity to grow the fragment in a specific and directed way to take advantage of the antibody-validated contact atoms and their positions in three dimensional space relative to the surface of the target cytokine and relative to the small molecule fragment.

One such interaction between the antibody Fab fragment and the target cytokine is between a first tyrosine (Tyr1) in the CDR3 heavy chain of the antibody and a tryptophan residue in the protein target, forming an edge-to-face pi-pi stacking interaction. Another interaction between the antibody Fab fragment and the target cytokine is a hydrogen bond between an asparagine (Asn1) in the CDR2 heavy chain region of the antibody Fab fragment and an arginine residue in the protein target. A further interaction is between a second tyrosine (Tyr2) in the CDR2 heavy chain region of the antibody and an arginine in the protein target which forms a cation-pi contact. The NCE fragment binds at a site within 4 Angstroms of these interactions, but is not able to take advantage of the three specific contacts because it lacks the appropriate structure at these position.

Analogues of the first fragment, which feature structural motifs to mimic the antibody interactions at the appropriate position relative to the core of the fragment may be preferentially selected for further analysis. Such analogues could involve, but are not limited to, a phenol ring to mimic Tyr1 or Tyr2 or a primary amide to mimic Asn1. Alternatively, new compounds may be specifically synthesized with a structure mimicking those interactions from the antibody in the appropriate positions. FIG. 1 shows how the fragment may be grown in this way, and how the new fragment may be expected to demonstrate enhanced binding through similar interactions observed with the antibody Fab fragment residues Tyr1, Tyr2 or Asn1.

FIG. 1a. Antibody Fab fragment (black backbone) with side chain residues Tyr1, Tyr2 and Asn1 (dark grey ball and stick) and the target protein (white molecular surface)

FIG. 1b. NCE fragment (dark grey molecular surface) and target protein (white molecular surface)

FIG. 1c. NCE fragment (dark grey molecular surface), target protein (white molecular surface) and antibody Fab fragment (black backbone)

FIG. 1d. NCE fragment has been grown to incorporate a phenol group as defined by Tyr1

FIG. 1e. NCE fragment has been grown to incorporate a phenol group as defined by Tyr2

FIG. 1f. NCE fragment has been grown to incorporate the oxygen and nitrogen atoms as defined by the Asn1 side chain.

FIG. 1g. NCE fragment has been grown to incorporate a phenol group as defined by Tyr1, a phenol group as defined by Tyr2 and the oxygen and nitrogen atoms as defined by the Asn1 side chain.

As the antibody has already validated both the additional chemical matter and its position relative to the existing fragment, the new candidate fragments generated by growing the compound fragment may be expected to have a higher probability of increased affinity and/or potency compared to growth by random substitution or by random selection from commercially available analogues.

Confirmation of increased binding or increased potency may be obtained by suitable assay, for example by BIAcore, FRET screening, or in an appropriate cell-based assay.

It is anticipated that the method will be iterative, in that new crystal structures of elaborated fragments will be obtained and compared to structures of antibody-target complexes, so that new opportunities for fragment growth and concomitant increased potency may be sought.

1. A method of generating a small molecule compound that can alter the activity of a target protein comprising:
(a) obtaining one or more antibodies or fragments thereof which bind to the target protein and alter the biological activity of the target protein
(b) generating a three-dimensional structural representation of an antibody obtained in step (a) in association with the target protein and identifying one or more pairs of contact atoms on the target protein and the antibody that interact with each other and which fall within a binding site of the antibody
(c) obtaining one or more compound fragments that bind to the target protein
(d) generating a three-dimensional structural representation of one or more of the fragments obtained in step (c) in association with the target protein
(e) selecting a compound fragment which binds the target protein within or in the vicinity of the antibody binding site identified in step (b)
(f) growing the compound fragment selected in step (e) to generate one or more candidate compounds which interact with one or more contact atoms on the target protein identified in step (b), optionally by directing the chemical growth of the compound fragment such that the expanded fragment occupies the same chemical space or 3D location as one or more of the contact atoms on the antibody identified in step (b)
(g) testing one or more of the candidate compounds produced in step (f) for improved affinity for the target protein and/or improved potency and/or the ability to alter the biological activity of the target protein
(h) selecting a compound tested in step (g) if it modulates the activity of the target protein or has improved binding affinity or ligand efficiency
(i) optionally performing further chemistry and screening using the compound identified in step (h) to generate a small molecule compound which modulates activity of the target protein.

2. The method according to claim 1 wherein each antibody or fragment thereof obtained in step (a) is selected from the group consisting of a complete antibody, a Fab, a modified Fab, a Fab′, Fv, VH, VL, VHH and an IgNAR V domain.

3. The method according to claim 1 wherein two or more antibodies are obtained in step (a).

4. The method according to claim 1 wherein at least one contact atom pair identified in step (b) consists of a contact atom on the target protein and a contact atom in the heavy chain or light chain variable region of the antibody that interact with each other.

5. The method according to claim 1 wherein at least one contact atom pair identified in step (b) consists of a contact atom on the target protein and a contact atom in a CDR of the antibody that interact with each other.

6. The method according to claim 1 wherein each contact atom pair identified in step (b) consists of a contact atom on the target protein and a contact atom in a CDR of the antibody that interact with each other.

7. The method according to claim 1 wherein at least one contact atom pair identified in step (b) consists of a contact atom on the target protein and a contact atom in a CDR of the heavy chain of the antibody that interact with each other.

8. The method according to claim 1 wherein two or more compounds are identified in step (c).

9. The method according to claim 1 wherein each compound fragment obtained in step (c) has a molecular weight of less than 500 Da.

10. The method according to claim 1 wherein the compound fragment selected in step (e) binds the target protein within 1-30 Å of a contact atom pair identified in step (b).

11. The method according to claim 1 wherein the three-dimensional structure generated in step (b) and/or step (d) is generated by X-ray crystallography.

12. A small molecule compound generated by the method of claim 1.
